(19) United States
(12) Patent Application Publication
(10) Pub. No.: US 2008/0140041 A1
(43) Pub. Date: Jun. 12, 2008

(54) Disposable absorbent undergarment

(76) Inventors: Eric Levis, New Caney, TX (US); Stacey Levis, New Caney, TX (US)

Correspondence Address: JAMES RAY & ASSOCIATES, 2640 PITCAIRN ROAD, MONROEVILLE, PA, 15146, US

(21) Appl. No.: 11/986,092

(22) Filed: Nov. 20, 2007

Related U.S. Application Data
(60) Provisional application No. 60/866,673, filed on Nov. 21, 2006.

(51) Int. Cl. A61F 13/15

(52) U.S. Cl. 604/385.13; 604/394; 604/385.01

(57) ABSTRACT

A disposable absorbent undergarment including a panty member, to receive a lower torso, buttocks and crotch of a user, the panty member having an inner surface, an outer surface, a front portion, a crotch portion, two side portions and a rear portion. The two side portions are elongate along a horizontal axis adjacent to the rear portion. The ends of the side portions are releasably secured to the outer surface. At least one absorbent layer is attached to the inner surface of the panty member. At least one liquid impervious layer is attached at a first side thereof to a bottom portion of the at least one absorbent layer and at a second side thereof to an inner surface of the panty member.

### CROSS REFERENCE TO RELATED APPLICATION

This patent application is related to and claims priority from U.S. Provisional Patent Application Ser. No. 60/866,673 filed Nov. 21, 2006.

### FIELD OF THE INVENTION

The present invention relates, in general, to feminine products and, more particularly, this invention relates to feminine products and underwear.

### BACKGROUND OF THE INVENTION

Prior to the conception and development of the present invention, feminine napkins as are generally well known in the prior art, have been used by women to protect underwear from becoming soiled due to menstruation. Specifically of interest to the present invention are the following: Ashton, et al, U.S. Pat. No. 7,074,215, discloses a slip-on disposable garment, primarily a diaper, including a chassis having a topsheet, a backsheet joined with the topsheet, and an absorbent core interposed between the topsheet and the backsheet. An elasticized waist structure is implemented for retaining the garment in its initial position about the waist of a user.

Ashton, et al, U.S. Pat. No. 6,626,879, discloses a disposable garment, primarily a diaper, adapted to be worn on the lower torso of a wearer. The garment includes a chassis including a topsheet, a backsheet joined with the topsheet, and an absorbent core interposed between the topsheet and the backsheet. An elasticized waist structure is implemented for retaining the garment in its initial position about the waist of a user.

Imsangjan, U.S. Pat. No. 7,011,653, discloses an absorbent pant garment (a diaper), including an absorbent chassis defining a waist opening and first and second leg openings. The absorbent chassis has a front region, a crotch region, and a back region, with two high cut side panels each joining the front region and the back region.

Rajala, U.S. Pat. No. 7,000,260, discloses a disposable garment, subassemblies of the garment, and apparatus and methods for making the garment and subassemblies. The garment has elasticized leg and waist openings and is similar to a diaper but claims to be for use by a menstruating female.

Although the above cited references indicate the claimed garments could be worn by a menstruating female, the claims truly read on diapers for infants and adults. None teach an undergarment similar enough to current standard undergarments that a menstruating female, a female experiencing heavy vaginal discharge or slight incontinence, or a female bleeding from childbirth and the like would actually be comfortable wearing in society, especially if the need arose for such female to disrobe is front of others, for example in a communal locker room of a gym or in front of her spouse or significant other.

Furthermore, none of the prior art teaches an item that can be folded into a small enough size and contained in a carrying case for easy storage and transportation in a user's purse, pocket, gym bag, and the like.

### SUMMARY OF THE INVENTION

The present invention provides a disposable absorbent undergarment including a panty member, manufactured from a first predetermined material, and having a first predetermined size, to receive a lower torso, buttocks and crotch of a user. The panty member has an inner surface, an outer surface, a front portion, a crotch portion, two side portions and a rear portion. The two side portions are elongate along a horizontal axis adjacent to said rear portion. A releasable means is attached to each of the side portions of the panty member. At least one absorbent layer is attached to an inner surface of the panty member, the absorbent layer being manufactured from a predetermined absorbent material and having a second predetermined size and a predetermined shape. At least one liquid impervious layer is attached at a first side thereof to a bottom portion of the at least one absorbent layer and at a second side thereof to the inner surface of the panty member.

The disposable absorbent under garment is foldable into a shape that is preferable and can be packed in a carrying case for easy transportation of the disposable absorbent under garment, such carrying case is disposable and may be used to enclose a used disposable absorbent undergarment for disposal.

### OBJECTS OF THE INVENTION

It is, therefore, one of the primary objects of the present invention to provide an absorbent disposable panty for a user to wear during menstruation or any other time a need for a protective undergarment arises.

Another object of the present invention is to provide an invention which fully protects a user's clothing from leaks and staining during times of menstruation, vaginal discharge, incontinence and bleeding associated with childbirth and the like.

Still another object of the present invention is to provide a comfortable undergarment for a user to wear during such user's menstruation period.

Yet another object of the present invention is to provide an inexpensive disposable undergarment that is more comfortable and provides more coverage and protection than a typical sanitary napkin, panty liner, or tampon used with an undergarment.

Still an additional object of the present invention is to provide a disposable panty for a user to wear that is capable of being folded and enclosed in a convenient disposable carrying case that can be carried easily in a purse, pocket, backpack and the like.

In addition to the various objects and advantages of the present invention described with some degree of specificity above it should be obvious that additional objects and advantages of the present invention will become more readily apparent to those persons who are skilled in the relevant art from the following more detailed description of the invention, particularly, when such description is taken in conjunction with the attached drawing figures and with the appended claims.

### BRIEF DESCRIPTION OF THE DRAWINGS

FIG. 1 is a front perspective view showing a disposable absorbent undergarment according to a first embodiment of the invention as worn by the user.

FIG. 2 is a rear perspective view showing a disposable absorbent undergarment according to a first embodiment of the invention as worn by the user.

FIG. 3 is a perspective view showing the interior surface of a disposable absorbent undergarment according to a first embodiment of the invention.

FIG. 4 is a view showing a disposable absorbent undergarment according to a first embodiment of the invention.

FIG. 5 is a top plan view showing a disposable absorbent undergarment according to one embodiment of the invention in which the undergarment is partially folded and retained in a carrying case.

FIG. 6 is a top plan view showing a disposable absorbent undergarment contained completely within a carrying case.

FIG. 7 is a front perspective view showing a disposable absorbent undergarment according to a second embodiment of the invention as worn by the user.

FIG. 8 is a perspective view showing a disposable absorbent undergarment according to a third embodiment of the invention as worn by the user.

FIG. 9 is a perspective view showing a disposable absorbent undergarment according to a fourth embodiment of the invention as worn by the user.

FIG. 10 is a perspective view showing a disposable absorbent undergarment according to a fifth embodiment of the invention as worn by the user.

### BRIEF DESCRIPTION OF A PRESENTLY PREFERRED AND VARIOUS ALTERNATIVE EMBODIMENTS OF THE INVENTION

Prior to proceeding to the more detailed description of the present invention it should be noted that, for the sake of clarity and understanding, identical components which have identical functions have been identified with identical reference numerals throughout the several views illustrated in the drawing figures.

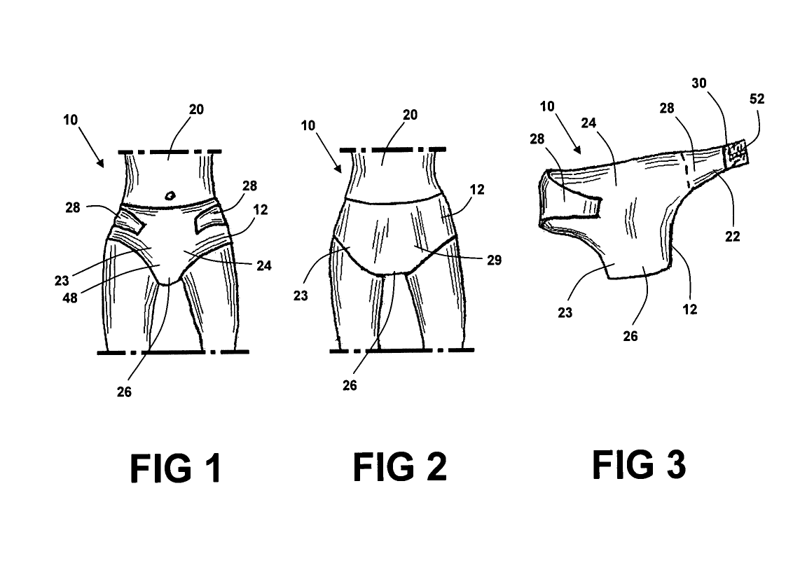

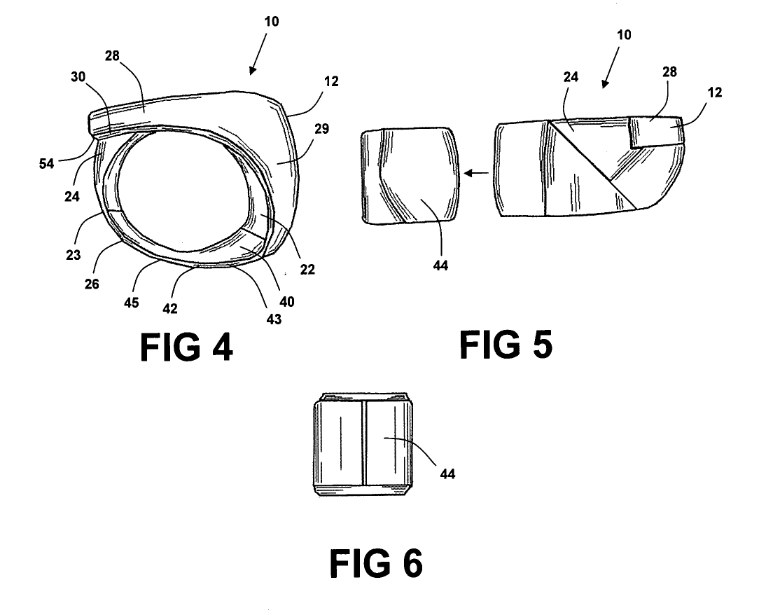

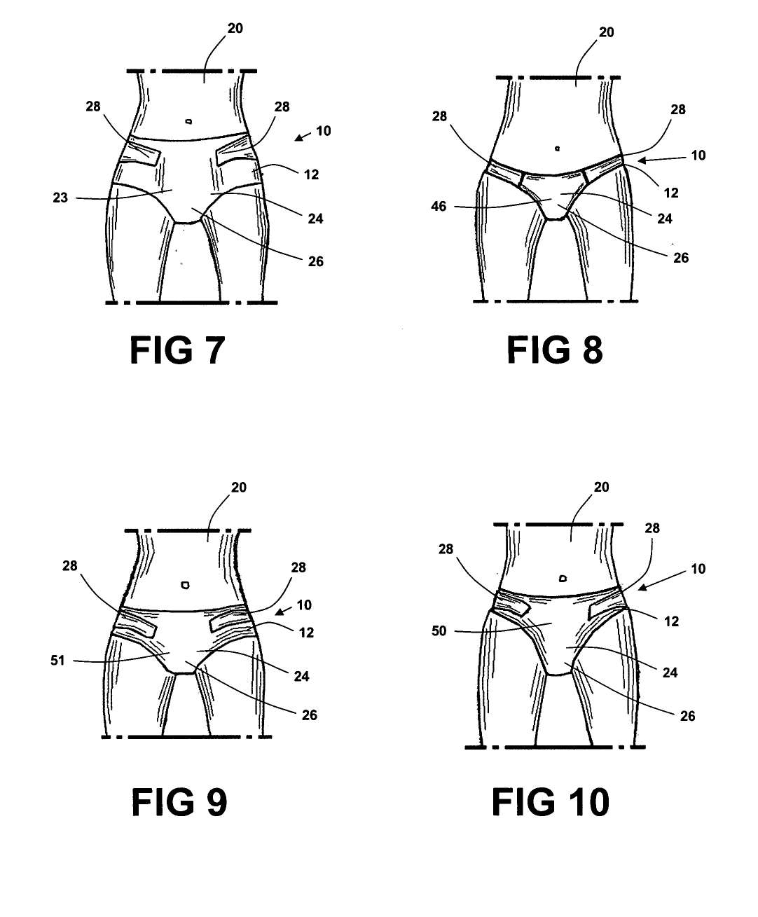

Reference is now made, more particularly, to FIGS. 1-10.

A disposable absorbent undergarment, generally designated 10, is provided. Such disposable absorbent undergarment includes a panty member 12, manufactured from a first predetermined material, and having a first predetermined size, and a predetermined shape and a predetermined thickness. Such panty member 12 functions to receive a lower torso, buttocks and crotch of a user 20. Such panty member 12 has an inner surface 22, an outer surface 23, a front portion 24, a crotch portion 26, two side portions 28 and a rear portion 29. The two side portions 28 are elongate along a horizontal axis adjacent to the rear portion 29.

A releasable means 30 is attached to each of the respective side portions 28 such that the side portions 28 are capable of being joined to the front portion 24.

At least one absorbent layer 40 is attached to the inner surface 22 of such panty member 12. The absorbent layer 40 being manufactured from a predetermined absorbent material and having a second predetermined size and a first predetermined shape and a predetermined thickness.

At least one liquid impervious layer 42 is attached at a first side thereof to a bottom portion 43 of such at least one absorbent layer 40 and at a second side 45 thereof to an inner surface 22 of such panty member 12.

As illustrated in FIGS. 5-6, in the presently preferred embodiment of the invention the disposable absorbent undergarment 10 is capable of being folded into a second predetermined shape of a third predetermined size and includes a disposable carrying case 44. It is further presently preferred that such disposable absorbent undergarment is prepackaged in such folded position within such disposable carrying case 44. After use the disposable absorbent undergarment 10 is foldable and can be placed in its carrying case 44 for disposal.

In the presently preferred embodiment of the invention, such panty member 12 is manufactured from an elastomeric material such that such panty member 12 is capable of being lowered from a user's body and capable of being pulled up onto such user's body from a lowered position without such user having to adjust said releasable means. It is presently preferred that such first predetermined material is at least one of cotton and nylon. It is presently most preferred that the first predetermined material is cotton. However, the predetermined material may also be various other materials known in the art for manufacture of undergarments.

It is further presently preferred that such panty member 12 is a least one of a bikini 46, brief 48, hi-cut brief 50, and low rider 51 and can be manufactured in a variety of colors and designs.

In the presently preferred embodiment of the invention, such releasable means 30 are hook and loop type fastening means 52. It is presently most preferred that such releasable means are adhesive type fastening means 54.

While a presently preferred and various alternative embodiments of the present invention have been described in sufficient detail above to enable a person skilled in the relevant art to make and use the same it should be obvious that various other adaptations and modifications can be envisioned by those persons skilled in such art without departing from either the spirit of the invention or the scope of the appended claims.

I claim:

1. A disposable absorbent undergarment comprising;
a. a panty member, manufactured from a first predetermined material, and having a first predetermined size, to receive a lower torso, buttocks and crotch of a user, said panty member having an inner surface, an outer surface, a front portion, a crotch portion, two side portions and a rear portion. Said two side portions are elongate along a horizontal axis adjacent to said rear portion;
b. a releasable means attached to each of said side portions of said panty member such that said side portions are capable of being joined to said front portion;
c. at least one absorbent layer attached to said inner surface of said panty member, said absorbent layer being manufactured from a predetermined absorbent material and having a second predetermined size and a first predetermined shape and a predetermined thickness; and
d. at least one liquid impervious layer attached at a first side thereof to a bottom portion of said at least one absorbent layer and at a second side thereof to said inner surface of said panty member.

2. A disposable absorbent undergarment according to claim 1 wherein said disposable absorbent undergarment is capable of being folded into a second predetermined shape of a third predetermined size.

3. A disposable absorbent undergarment according to claim 2 wherein said disposable absorbent undergarment further includes a disposable carrying case.

4. A disposable absorbent undergarment according to claim 1 wherein said panty member is a least one of a bikini, brief, hi-cut brief, and low rider.

5. A disposable absorbent undergarment according to claim 1 wherein said first predetermined material is selected from the group consisting of cotton and nylon.

6. A disposable absorbent undergarment according to claim 1 wherein said first predetermined material is an elastomeric material such that said panty member is capable of being lowered from a user's body and capable of being pulled up onto such user's body from a lowered position without such user having to adjust said releasable means.

7. A disposable absorbent undergarment according to claim 1 wherein said releasable means are hook and loop type fastening means.

8. A disposable absorbent undergarment according to claim 1 wherein said releasable means are adhesive type fastening means.

9. A disposable absorbent undergarment according to claim 1 wherein said panty member can be manufactured in a variety of colors and designs.

10. A disposable absorbent undergarment according to claim 1 wherein said disposable absorbent undergarment is prepackaged in a folded position within a disposable carrying case.
